(19) United States
(12) Patent Application Publication
(10) Pub. No.: US 2010/0121179 A1
(43) Pub. Date: May 13, 2010

(54) Systems and Methods for Reducing RF Power or Adjusting Flip Angles During an MRI for Patients with Implantable Medical Devices

(75) Inventor: Xiaoyi Min, Thousand Oaks, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/270,768

(22) Filed: Nov. 13, 2008

(51) Int. Cl. A61B 5/055

(52) U.S. Cl. 600/421; 600/410

(57) ABSTRACT

Techniques are provided for controlling magnetic resonance imaging (MRI) systems for imaging patients having implantable medical devices. In one example, a scaling factor is determined based on maximum local specific absorption rate (SAR) values for patients with implants and for patients without implants. The MRI determines the radio-frequency (RF) power and flip angle sequences to be used for a given patient, without regard to the presence of an implanted device. However, for patients with implanted devices, the MRI reduces its RF power or adjusts its flip angle sequences based on the scaling factor so as to ensure that the local SAR within the patient does not exceed acceptable levels. In other examples, rather than reducing the RF power of the MRI or adjusting the flip angles, blankets or pads formed of RF power attenuating materials, such as dielectrics, are positioned around the patient near the implantable device, to reduce the RF power incident tissues adjacent the device.

### FIELD OF THE INVENTION

The invention generally relates implantable medical devices, such as pacemakers or implantable cardioverter-defibrillators (ICDs), and to magnetic resonance imaging (MRI) procedures and, in particular, to techniques for preventing damage to implantable devices and patient tissues during an MRI.

### BACKGROUND OF THE INVENTION

MRI is an effective, non-invasive magnetic imaging technique for generating sharp images of the internal anatomy of the human body, which provides an efficient means for diagnosing disorders such as neurological and cardiac abnormalities and for spotting tumors and the like. Briefly, the patient is placed within the center of a large superconducting magnetic that generates a powerful static magnetic field. The static magnetic field causes protons within tissues of the body to align with an axis of the static field. A pulsed radio-frequency (RF) magnetic field is then applied causing the protons to begin to precess around the axis of the static field. Pulsed gradient magnetic fields are then applied to cause the protons within selected locations of the body to emit RF signals, which are detected by sensors of the MRI system. Based on the RF signals emitted by the protons, the MRI system then generates a precise image of the selected locations of the body, typically image slices of organs of interest. With an MRI system, both the power of the RF fields and the flip angle of the magnetic fields can be adjusted. The flip angle is the angle by which a net magnetization vector is rotated away from that of the main magnetic field during the application of an RF pulse. Flip angle is sometimes also referred to as the tip angle, nutation angle or angle of nutation.

However, MRI procedures are problematic for patients with implantable medical devices such as pacemakers and ICDs. One of the significant problems or risks is that the strong RF fields of the MRI can induce currents through the lead system of the implantable device into the tissues resulting in Joule heating in the cardiac tissues around the electrodes of leads, potentially damaging adjacent tissues. Indeed, in worst case scenarios, the temperature at the tip of an implanted lead has been found to increase as much as 70 degrees Celsius (C.) during an MRI tested in a gel phantom in a non-clinical configuration. Although such a dramatic increase is probably unlikely within a clinical system wherein leads are properly implanted, even a temperature increase of only about 80-13° C. might cause myocardial tissue damage.

Furthermore, any significant heating of cardiac tissues near lead electrodes can affect the pacing and sensing parameters associated with the tissues near the electrode, thus potentially preventing pacing pulses from being properly captured within the heart of the patient and/or preventing intrinsic electrical events from being properly sensed by the device. The latter might result, depending upon the circumstances, in therapy being improperly delivered or improperly withheld. Another significant concern is that any currents induced in the lead system can potentially generate voltages within cardiac tissue comparable in amplitude and duration to stimulation pulses and hence might trigger unwanted contractions of heart tissue. The rate of such contractions can be extremely high, posing significant clinical risks on patients. Therefore, there is a need to reduce heating in the leads of implantable medical devices, especially pacemakers and ICDs, and to also reduce the risks of improper tissue stimulation during an MRI, which is referred to herein as MRI-induced pacing.

A variety of techniques have been developed for use with implantable devices and their leads to reduce the adverse affects of MRI fields, such as installing RF filters or switches within the leads for filtering signals associated with the RF fields of MRIs to reduce the effect of such signals on the device and on patient tissues. Nevertheless, MRI scans are still contraindicated for patients with active implantable medical devices (AIMD), such as pacemakers and ICDs, due to the risks of force/torque from static fields, potential stimulation from gradient fields, and heating from RF fields. Indeed, validation standards have not yet even been established by the U.S. Food and Drug Administration (FDA) for validating the use of MRI systems on patients with AIMDs. Validation is complicated by the wide variety of AIMDs that might be implanted within patients, including the many different combinations and orientations of leads used with such devices. Accordingly, it would be highly desirable to provide systems and methods for allowing validation of the use of MRI systems on patients with AIMDs that does not require separate validation for different combinations of MRI systems, AIMDs and lead arrangements, and it is to this end that certain aspects of the invention are directed.

Although the FDA has not yet established a validation protocol for the use of MRIs on patients with AIMDs, the ISO/IEC and FDA have worked on developing an appropriate tiered strategy, which is to establish the worst case conditions for an entire patient population and for all MRI systems. The worst case conditions are to be determined using human body models with RF coils for deriving conclusions for an entire patient population. Actual implantable devices must then be tested in a gel phantom at the worst case condition, with sufficient validations performed and with confidence obtained in computer models. Due to the many variables arising among different patients, different MRI systems and different implantable devices, establishing the worst case condition is a challenging task.

One possible method is to accurately model the fields and absolute temperatures generated within the tissues of the patient at local specific absorption rate (SAR) limits (or max B1 rms (root mean square) limits) in human models to ascertain worst-case heating conditions. SAR is a measure of the rate at which RF energy is absorbed by bodily tissues when exposed to RF fields, i.e. SAR=σ*|E|²/ρ where ρ is mass density and σ is electrical conductivity of tissue. SAR is proportional to |E(r)|². The whole-body SAR is the average of the local SAR over the human body. The local SAR limit is the maximum local SAR allowed in the human body during an MRI scan. (It is known that the whole-body SAR is not a good measure for RF heating due to inconsistencies among different MRI systems. Hence, local SAR is preferably modeled.)

Modeling the absolute temperature generated by an actual MRI system at local SAR limits and then accessing the worst-case heating condition is a very challenging task. Hence, it would be desirable to provide systems and methods for allowing the validation of MRI systems on patients with AIMDs that do not rely on extensive modeling and measurements and which instead employs a simpler strategy, and it is to this end that other aspects of the invention are directed.

Setting aside issues of validation, it would also be desirable to provide systems and methods for improving or ensuring the safety of patients with AIMDs when undergoing MRIs and still other aspects of the invention are directed to this general goal.

### SUMMARY OF THE INVENTION

In accordance with a first general embodiment of the invention, systems and methods are provided for controlling MRI systems for safely imaging the tissues of patients with implantable medical devices, particularly AIMDs. Briefly, the systems and methods exploit a scaling factor derived from predetermined SAR values for use in reducing the RF power of the MRI and/or for adjusting the flip angle of the MRI to reduce incident power within the tissues of the patient. In use, the MRI system initially determines appropriate RF power levels and flip angle sequences for a particular patient to be imaged without regard to the presence of the implantable medical device in the patient. The MRI system then reduces RF power levels and adjusts flip angles using the scaling factor prior to imaging. With proper selection of the scaling factor, heating within the patient remains at safe levels during the MRI despite the presence of the implantable device. Also, with proper selection of the scaling factor, any MRI system validated for use on patients without implants likewise qualifies for validation on patients with implants, thus obviating the need to separately or individually validate MRI systems with different implantable devices, lead combinations, etc.

In one example, a predetermined maximum SAR value (maxSARo) for patients without implantable devices is input into the MRI system. The maxSARo value represents the maximum permissible or allowable SAR value that can safely be generated within patients without implants. This value is specified by the FDA or other appropriate government authority. A predetermined maximum SAR value (maxSARi) for patients with implantable devices is also input. The maxSARi value represents the corresponding SAR value that would result in tissues of a patient with an implant due to the presence of that implant. This value, which is larger than maxSARo, is initially determined by, e.g., MRI system manufacturers. When a patient with an implant needs an MRI, an operator of the MRI system controls the MRI system to enter a user-activated Implant Mode wherein the MRI uses a scaling factor to reduce its RF power and/or to adjust its flip angles to account for the implant. The scaling factor is determined based on maxSARo and maxSARi and, in one example, the scaling factor is the ratio of maxSARo/maxSARi, referred to herein as R_{SAR}. With maxSARo less than maxSARi, the scaling factor R_{SAR} is therefore less than 1.0. The P_{RF} value to be used is reduced based on the scaling factor. Alternatively, flip angles or flip angle sequences are adjusted to achieve a corresponding or equivalent reduction in RF power incident patient tissues. Selected tissues of the patient are then imaged using the MRI system at the reduced power level and/or with the adjusted flip angles. Preferably, the maximum and minimum SAR values are local SAR values corresponding to particular tissues to be imaged, such as thoracic tissues for the case of patients with pacemakers and ICDs.

In practice, the MRI system initially determines an initial or baseline P_{RF} value for the particular patient to be imaged while assuming no implant is present. The initial P_{RF} value may be determined using conventional or proprietary MRI techniques that have been validated for use with patients without implants. The P_{RF} value is then reduced by the scaling factor before imaging the patient with the implant (or the flip angle is adjusted.) In one particular example, P_{RF} is reduced by multiplying P_{RF} by R_{SAR}, i.e. P_{RF}_{_{—}}_{NEW}=P_{RF}*R_{SAR} so as reduce P_{RF} by the R_{SAR} ratio to account for the implant. In this manner, procedures and algorithms already validated by the FDA can be used by the MRI system to initially determine the P_{RF} value for the patient (without regard to the presence of the implanted device.) Then, the P_{RF} value is reduced by applying the R_{SAR} ratio, yielding a new, lower P_{RF} value that will be safe, despite the presence of the implantable device.

This technique exploits the recognition that the relationship between P_{RF} and maximum SAR is linear and proportional. That is, the RF power input to the tissues of a patient from the RF coils of an MRI system is assumed to be equal to the total energy dissipated in the patient, i.e. P_{RF}=I·V=∫_{V} σ*E*E dV, which is proportional to the whole-body SAR, where I is the total current, V is the voltage from RF coils, E is the energy and σ represents tissue electrical conductivity which varies with the tissues in human body. Since the electromagnetic fields generated in the patient are linear to both the current I and the voltage V, the SAR for the patient is likewise linearly proportional to P_{RF}. Accordingly, P_{RF} can be scaled linearly based on SAR values. In particular, P_{RF} can be scaled linearly based on the ratio of maxSARo to maxSARi. That is, assuming that the P_{RF} determined by an FDA-approved MRI system yields a SAR value (within a patient without an implant) that does not exceed maxSARo, then a P_{RF} value reduced by R_{SAR} will likewise yield a SAR value (within a patient with an implant) that does not exceed maxSARo. Similar considerations apply to the reduction in incident power achieved via changes in flip angle.

Hence, any MRI system validated for use by the FDA (or other appropriate government entity) on patients without implants should likewise qualify for validation on patients with implants, assuming P_{RF} is scaled by R_{SAR} or the flip angle is properly adjusted based on R_{SAR}, thus obviating the need to separately or individually validate MRI systems with different implantable devices, lead combinations, etc. At least, any MRI system validated for use on patients without implants should be more easily validated for use on patients with implants, when exploiting these SAR-based techniques.

Insofar as the initial determination of maxSARo and maxSARi is concerned, maxSARo is specified, as noted, by the FDA or other appropriate government entity and is typically set, e.g., to 10 watts (W)/kilogram (kg) for MRI procedures. MRI systems must be set such that they do not exceed this SAR value within patients without implants. The value for maxSARi may be determined by MRI system manufacturers through suitable modeling in human body models and experimentation using gel phantoms or cadavers equipped with clinically relevant “worst case” implant configurations with “worst case” MRI configurations. That is, the maxSARi value may be determined for the worst case (maxSARi_max), thereby yielding a scaling factor that likewise represents the worst case (R_{SAR}_{_{—}}_{MAX}), hence ensuring that the P_{RF} value to be used on patients with implants will be reduced sufficiently to ensure patient safety, even in worse case situations.

By exploiting the worst case scenario, modeling and/or experimental validations are no longer required to ascertain maxSARi for all MRI systems or to determine different maxSARi values associated with each individual MRI system. However, in some implementations, it may be desirable to further specify different maxSARi values for use with different MRI machines, such that a unique R_{SAR} value is determined for use with each MRI machine. For example, one particular R_{SAR} value is determined for use with MRI machine A provided by Manufacturer A; whereas a different R_{SAR} value is determined for use with MRI machine B provided by Manufacturer B. Likewise, in some implementations, it may be desirable to further specify different maxSARi values for use with different implantable devices and leads, such that a unique R_{SAR} value is determined for use with each model of implantable device or each model of device lead. For example, one particular R_{SAR} value is determined for use with Pacemaker C provided by Manufacturer C; whereas a different R_{SAR} value is determined for use with Pacemaker D provided by Manufacturer D. That is, rather than determining R_{SAR} based on the single worst case scenario, a plurality of R_{SAR} values are obtained for use in different circumstances. The information can be exploited in the “Implant Mode” of the MRI machines. These added levels of specificity permit generally higher RF power levels to be used in most cases so as to provide better MRI images, while still ensuring patient safety.

In any case, once MRI systems have been validated for use with patients with implants, an individual MRI system can then: determine an initial P_{RF} value and flip angle based on a particular patient to be imaged without regard to the presence of the implantable medical device within the patient; input or determine the R_{SAR} ratio that is appropriate; scale the P_{RF} value based on the ratio or adjust the flip angle to achieve the same amount of power reduction; and then image the tissues of the patient at the reduced power levels.

In another example of the first general embodiment of the invention, when a patient with an implant needs an MRI, the implanted device detects the MRI system and switches into an MRI Mode of operation. Once in the MRI Mode, the device transmits a signal to the MRI system to notify the MRI system of the implant and to control the MRI system to automatically enter its Implant Mode. That is, rather than having a “user-activated” Implant Mode, the MRI system has an automatic “device-activated” Implant Mode. In this mode, the implanted device can additionally transmit information specifying the make/model of the implanted device and the make/model of the lead system for use in setting MRI imaging parameters. Also, the implanted device can transmit information identifying any MRI-responsive features of the implanted device.

When using the device-activated Implant Mode of the MRI system, rather than using a maxSARi value validated for general patient populations, a maxSARi value can instead be employed that takes into account specific attributes of the particular medical device implanted in the patient to be imaged. The use of these “device-specific” maxSARi values may be helpful in allowing the MRI system to use more RF power than would be permitted based on a general “worst case” maxSARi, thus yielding higher resolution images in at least some patients. For example, implantable devices are increasingly equipped with RF filters or other devices for mitigating the effects of MRI fields. Patients with such devices can be safely imaged with stronger RF fields than would be used if using a general “worst case” maxSARi to scale the RF power. Accordingly, information pertaining to particular makes/models of devices that might be implanted within patients is programmed into the MRI system in advance. The MRI system then uses the stored information in conjunction with the information transmitted from the device to determine the appropriate scaling factor to be used for that patient.

In yet another example of the first general embodiment of the invention, the MRI Mode of the implanted device operates to transmit signals to the MRI system providing patient-specific data (typically in addition to device-specific data.) The patient-specific data can include a predetermined maximum SAR value for the particular patient or the appropriate flip angle to be used when imaging the particular patient. The Implant Mode of the MRI system then exploits the patient-specific data to set the RF power, flip angle, etc., of the imaging fields. Alternatively, rather than storing patient-specific or device-specific data within the pacer/ICD, such information can be stored within the MRI system. That is, the MRI machine stores all the information needed such as R_{SAR} and flip angle associated with the scaling factor. In this mode, the choices for different device manufactures are shown on one display screen and further selection of device specifics is made in a subsequent screen.

In accordance with a second general embodiment of the invention, rather than reducing the power of the RF fields of the MRI system for patients with implants, RF power attenuation materials, such as blankets, jackets or pads containing suitable dielectric or resistive/conductive materials, are instead placed around the patient, particularly around the portions of the patient in which devices are implanted. For example, blankets or pads containing dielectric or resistive/conductive materials may be wrapped around the chest of a patient with a pacemaker or ICD. The RF power attenuation materials reduce the RF power radiating patient tissues in the vicinity of the implantable device by an amount sufficient to ensure that maxSARo is not exceeded within those tissues. In some implementations, different articles/materials with different thicknesses are tested and validated in advance to achieve different reductions in RF power within patient tissues. MRI personnel then select the particular RF power attenuation articles/materials that are appropriate for a given patient similar to the information input into MRI before scans such as patient weight etc. Patient-specific attributes, such as whether the medical devices implanted therein are equipped with RF filters, may also be taken into account when selecting the articles/materials to be used. For example, blankets of differing thickness may be provided. MRI personnel then select the appropriate thickness for use with a given patient based, in part, on the attributes of the implanted medical device or other patient attributes.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 is a stylized representation of a first exemplary MRI system, along with a patient with a pacer/ICD implanted therein, wherein the MRI system is equipped with a user-activated Implant Mode for use with patients with implantable devices to reduce RF power and/or adjust flip angles for patients with implantable devices;

FIG. 2 is a flow diagram providing an overview of techniques performed in conjunction with the MRI system of FIG. 1 for reducing the power of the RF fields and/or adjusting flip angles;

FIG. 3 is a flow diagram providing a more detailed illustration of exemplary processing techniques in accordance with the procedure of FIG. 2, illustrating differing operation depending upon whether or not the patient being imaged has an implantable device;

FIG. 4 is a stylized representation of a second exemplary MRI system, wherein the MRI is equipped with a device-activated Implant Mode that receives and stores device-specific data to aid in setting RF power and flip angles to optimal values;

FIG. 5 is a flow diagram providing an overview of device-specific operational techniques performed by the pacer/ICD and MRI system of FIG. 4 for reducing the power of the RF fields;

FIG. 6 is a stylized representation of a third exemplary MRI system, wherein the MRI system is equipped with a device-activated Implant Mode that receives and stores patient-specific data from the pacer/ICD to aid in setting RF power and flip angles to optimal values;

FIG. 7 is a flow diagram providing an overview of the patient-specific operational techniques performed by the pacer/ICD and MRI system of FIG. 6 for reducing the power of the RF fields;

FIG. 8 is a stylized representation of another exemplary MRI system, wherein RF power attenuation materials are placed around the patient during an MRI procedure to reduce the power of RF fields radiating the tissues of the patient around the pacer/ICD;

FIG. 9 is a flow diagram providing an overview of the technique of using RF power attenuation materials for reducing the power of RF fields incident the patient, as in the system of FIG. 8;

FIG. 10 is a flow diagram providing a more detailed illustration of exemplary processing techniques in accordance with the general procedures of FIG. 9, wherein particular RF power attenuation materials are selected based on a scaling factor derived from maxSARi and maxSARo values;

FIG. 11 is a simplified, partly cutaway view, illustrating the pacer/ICD of FIG. 6 along with a full set of leads implanted in the heart of the patient;

FIG. 12 is a functional block diagram of the pacer/ICD of FIG. 11, illustrating basic circuit elements that provide cardioversion, defibrillation and/or pacing stimulation in four chambers of the heart and particularly illustrating components for storing and transmitting patient-specific and device-specific data.

### DETAILED DESCRIPTION OF THE PREFERRED EMBODIMENTS

The following description includes the best mode presently contemplated for practicing the invention. The description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of MRI System With User-Activated Power-Scaling Mode

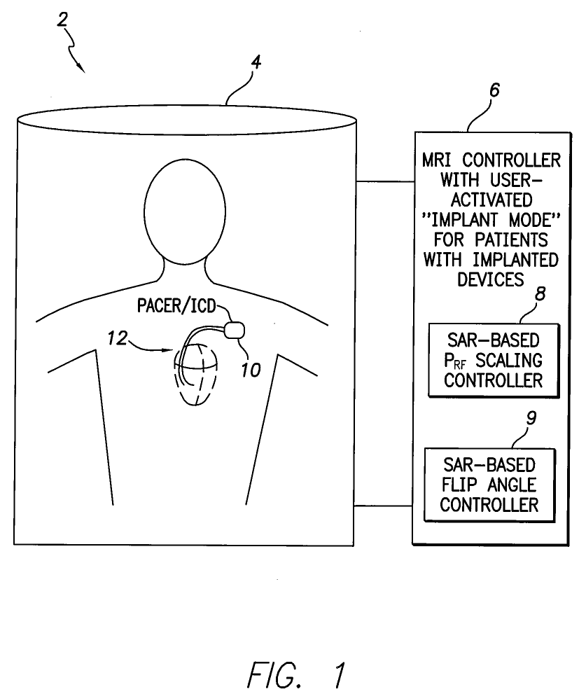

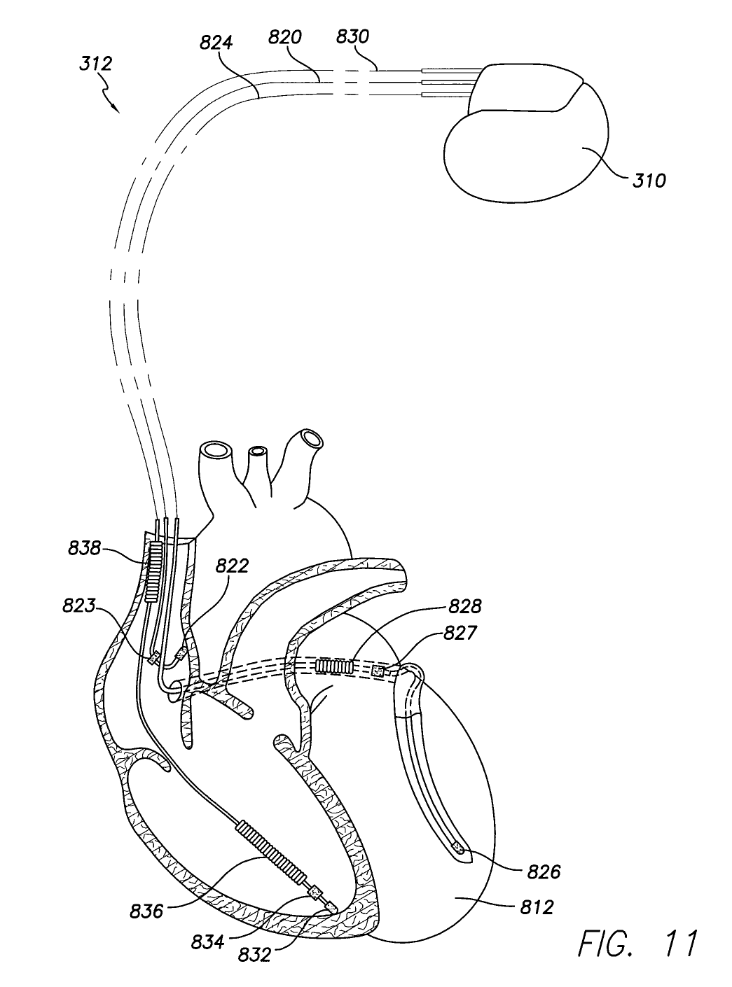

FIG. 1 illustrates an MRI system 2 having an MRI machine 4 operative to generate MRI fields during an MRI procedure for examining a patient. The MRI machine operates under the control of an MRI controller 6, which controls the strength and orientation of the fields generated by the MRI machine and derives images of portions of the patient therefrom, in accordance with otherwise conventional techniques. MRI systems and imaging techniques are well known and will not be described in detail herein. See, for example, U.S. Pat. No. 5,063,348 to Kuhara, et al., entitled “Magnetic Resonance Imaging System” and U.S. Pat. No. 4,746,864 to Satoh, entitled “Magnetic Resonance Imaging System.” Also shown is a pacer/CD 10 implanted within a patient being imaged during the MRI procedure. A lead system 12 is coupled to the pacer/ICD for sensing electrophysiological signals within the heart of the patient and for delivering any needed pacing pulses or shock therapy. In FIG. 1, only two leads are shown. A more complete lead system is illustrated in FIG. 11, described below.

MRI controller 6 is equipped to exploit a user-activated Implant Mode for patients with implantable devices. As will be described below, other implementations of the MRI system operate to automatically detect the presence of an implantable medical device within the patient via notification signals received from the implantable device to thereby activate the Implant Mode.

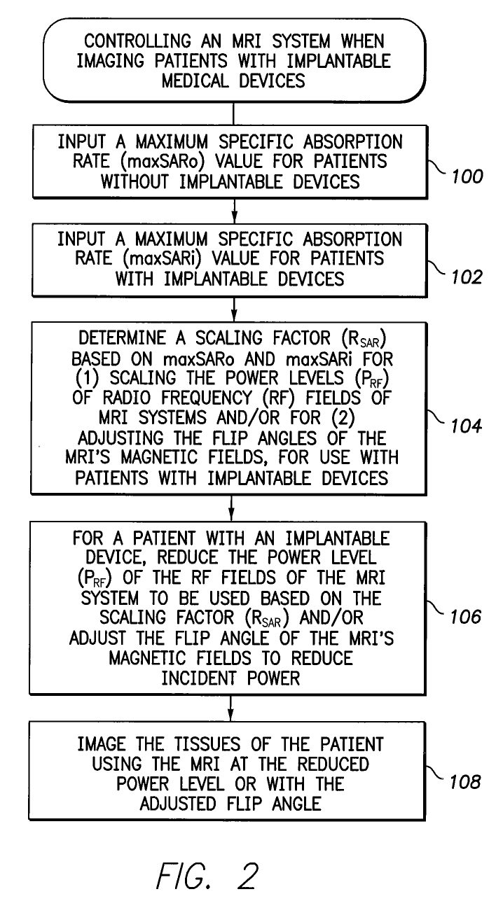

In the example of FIG. 1, the Implant Mode of the MRI controller exploits a SAR-based P_{RF} scaling controller 8, which is operative to adjust the power of the RF fields of MRI system 4. That is, upon activation by a user (i.e. by the operator of the MRI), the scaling controller determines an initial or baseline P_{RF} value based on the particular patient to be imaged without regard to the presence of pacer/ICD 10. The scaling controller also inputs an R_{SAR} ratio (pre-determined using techniques to be described in FIG. 2.) Then, the scaling controller reduces or scales the P_{RF} value based on the R_{SAR} ratio before imaging the tissues of the patient at the reduced power level.

In this example, the Implant Mode of the MRI controller also exploits a SAR-based flip angle controller 9, which is operative to adjust the slip angle of the magnetic fields of the MRI. That is, upon activation, the flip angle controller determines an initial or baseline flip angle or flip angle sequence based on the particular patient to be imaged without regard to the presence of pacer/ICD 10. The flip angle controller also inputs an R_{SAR} ratio (pre-determined using techniques to be described in FIG. 2.) Then, the scaling controller then adjusts the initial flip angle based on the R_{SAR} ratio before imaging the tissues of the patient. The flip angle is adjusted to achieve the same effective amount of RF power reduction within the tissues of the patient as if P_{RF} were directly reduced. In some implementations, P_{RF} and flip angle are both adjusted to collectively achieve the appropriate amount of power reduction within the tissues of the patient. In other implementations, the MRI controller does not include both a SAR-based power scaling controller and a SAR-based flip angle controller.

### SAR-Based RF Power-Scaling/Flip Angle Adjustment Procedures

FIG. 2 broadly summarizes techniques for determining the R_{SAR} scaling factor and for controlling an MRI machine, such as the one of FIG. 1, when imaging patients with implants. Beginning at step 100, a maximum local SAR value (maxSARo) for patients without implantable devices is input into the MRI controller by users or programmers or the MRI system. The maxSARo value is determined and validated in advance. Typically, this value is specified by government agencies. For example, in the U.S., the FDA specifies the maximum local SAR value for MRI procedures for patients without implantable devices and so, at step 100, that maximum value is simply input into the scaling controller by personnel operating or programming the MRI system. The current local maxSARo value specified by the FDA is 10 W/kg for MRI procedures. In other countries, this value might be different. The appropriate value for the jurisdiction in which the MRI system is being used should be employed. If no maximum local SAR value has already been specified, otherwise conventional experiments may be performed using various MRI systems and various test phantoms (or other test models) without implants to determine and validate a suitable value for maxSARo. Also note that, in some jurisdictions, different maxSARo values might be specified for different populations of patients, such as different age ranges, genders, or for different tissues within the patient, etc. If so, then the appropriate maxSARo value should be input into the scaling controller at step 100 based on the particular patient to be imaged. (In such circumstances, a table of maxSARo values may be pre-stored in the scaling controller, then the pertinent characteristics of the patient to be imaged are input so that the scaling controller can select the appropriate maxSARo value for use with the patient.)

At step 102, a maximum local SAR value (maxSARi) for patients with implantable devices is input into the MRI controller by users or programmers or the MRI system. The maxSARi value is also determined in advance. Currently, this value is not specified by the FDA or other government agencies of the U.S. As such, experiments and tests are performed at step 102, preferably by MRI system manufacturers, to determine an appropriate value for maxSARi by using various MRI systems and various test phantoms or human body models (with implants) to validate the value. Preferably, the same (or similar) techniques originally used to ascertain and validate maxSARo are also used to ascertain and validate maxSARi, but applied to phantoms (or other test models) with implants, rather than phantoms without implants.

Preferably, the maxSARi value determined is a worst case value. Once the FDA or other appropriate government entity has accepted and validated the maxSARi value, the value is then input into the scaling controller. If the MRI system is used in foreign jurisdictions that have specified a different maxSARi value, the appropriate value for the jurisdiction in which the MRI system is being used should be employed. As with maxSARo, in some jurisdictions, different maxSARi values might be specified for different populations of patients, such as different age ranges, genders, or for different tissues, etc. If so, then the appropriate maxSARi value should be determined for use at step 102. (Again, a table of maxSARi values may be stored in the scaling controller, then the pertinent characteristics of the patient to be imaged are input so that the scaling controller can then select the appropriate maxSARi value for use with the patient.)

At step 104, the MRI controller then determines (or inputs) the R_{SAR} scaling factor for scaling the power levels (P_{RF}) of RF fields of the MRI system for use with a patient with an implantable devices based on the input values for maxSARo and maxSARi and/or for adjusting the flip angle. Preferably, R_{SAR} is calculated as the ratio of maxSARo/maxSARi. As such, if maxSARo is 10 W/kg and maxSARi is determined to be 12 W/kg, then R_{SAR} is 0.8333. If different maxSARi and maxSARo values are specified for different populations of patients, then R_{SAR} is calculated based on the particular maxSARi and maxSARo values appropriate for the particular patient. (Assuming that a “worst case” value for maxSARi was determined at step 102, then the R_{SAR} value calculated at step 104 may also be specifically referred to as R_{SAR}_{_{—}}_{MAX}.) At step 106, for a particular patient to be imaged, the scaling controller reduces the power level (P_{RF}) of the RF fields of the MRI machine that would otherwise be used based on the scaling factor. For example, P_{RF} is reduced by multiplying P_{RF} by R_{SAR}, i.e. P_{RF}_{_{—}}_{NEW}=P_{RF}*R_{SAR}.

Alternatively, the MRI system adjusts its flip angle or flip angle sequences at step 104 to achieve a corresponding or equivalent reduction in incident power within the tissues of the patient. Insofar as flip angle adjustment is concerned, the precise manner in which flip angle(s) or flip angle sequences are adjusted by the MRI system to achieve an equivalent amount of power reduction will depend upon the particular imaging software and systems of the MRI system, which are typically proprietary. Those skilled in the art of MRI system design can, without undue experimentation, determine how to modify MRI systems and software to adjust flip angle(s) and flip angle sequences to achieve equivalent power reduction based on the scaling factor. That is, the MRI systems and software are pre-programmed to input the scaling factor and to automatically adjust flip angle(s) and flip angle sequences (and any other parameters that might require adjustment depending upon the particular MRI system.) In any case, from the standpoint of the user or operator of the MRI, these adjustments are made automatically by the MRI system based on the scaling factor prior to imaging the patient.

At step 108, the MRI system then images the tissues of the patient using the MRI at the reduced power level or with the adjusted flip angle. (As a practical matter, of course, any necessary government approval/validation should be obtained before using the modified MRI system to image a patient with an implant.)

In this manner, MRI controller 6 of FIG. 1 can use procedures and algorithms previously validated by the FDA (or other appropriate government entities) to initially determine the appropriate P_{RF} value for the patient, without regard to the presence of the implanted device. Then, the P_{RF} value is reduced by the R_{SAR} ratio, yielding a new P_{RF} value or flip angle that will be safe for the patient, despite the presence of the implantable device. As summarized above, this scaling technique exploits the recognition that the relationship between P_{RF} and maximum SAR is linear and proportional. The RF power input to the tissues of a patient from the MRI system is assumed to be equal to the total energy dissipated in the patient, i.e. P_{RF}=I·V=∫_{V} σ*E*E dV, which is proportional to the whole-body SAR. Since the electromagnetic fields generated in the patient are linear to both I and V, the SAR for the patient is likewise linearly proportional to P_{RF}. Accordingly, P_{RF} can be scaled linearly by the scaling controller of the MRI system based on the ratio of maxSARi to maxSARo. That is, assuming that the P_{RF} value determined by MRI controller 6 yields SAR values within patients without implants that do not exceed maxSARo within those patients, then a P_{RF} value reduced by the ratio R_{SAR} will likewise yield a SAR value within the patient with implant 10 that that does not exceed maxSARi for that patient. Suitable adjustments in flip angle will provide the same results.

As such, MRI system 2 (or any other MRI system validated for use by the FDA or other government entities for use with patients without implants) should likewise be safe for use with patients with implants, assuming P_{RF} is scaled by R_{SAR} or the flip angle is properly adjusted to achieve the same result. Accordingly, MRI system 2 should qualify for government approval for use with patients with implants, again assuming P_{RF} is scaled by R_{SAR}, thus obviating the need to separately or individually validate MRI system 2 with different implantable device models, different lead combinations, etc. At the very least, any MRI system validated for use on patients without implants should more readily be approved for use on patients with implants, when exploiting the power-scaling technique of FIG. 2, as compared to validation techniques of the type discussed above in the Background section where the absolute temperatures generated by an MRI system within the tissues of a patient are modeled at local SAR limits to access worst-case heating conditions. Hence, the costs and time delays associated with validation can be greatly reduced, while also helping to ensure patient safety.

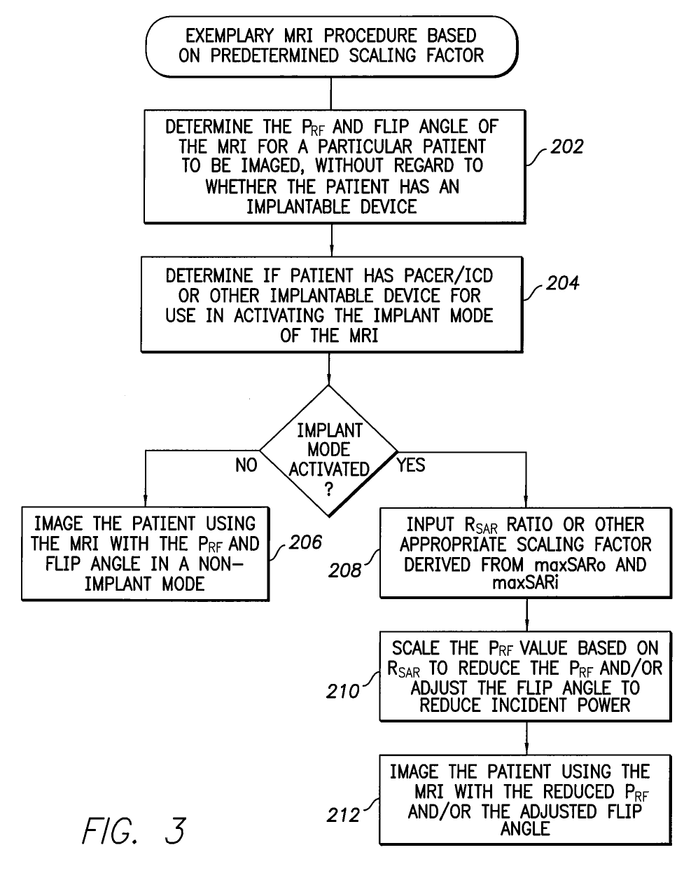

FIG. 3 summarizes the steps of an individual imaging procedure. At step 202, under the control of an operator or user, the MRI controller (i.e. controller 6 of FIG. 1) determines the P_{RF} and flip angle of the MRI for the patient to be imaged, without regard to whether the patient has an implantable device. This may be performed in accordance with otherwise conventional or proprietary techniques that have already been approved/validated by appropriate government authorities. At step 204, the operator of the MRI system then determines if the patient has a pacer/ICD or other implantable device and inputs that information into the MRI system to activate or enter the Implant Mode. (In some implementations, discussed below, the MRI system may be equipped to automatically detect whether the patient has an implantable device based on signals transmitted from the device.)

Assuming the patient does not have an implantable device, then, at step 206, then the Implant Mode is not activated by the user and the MRI system images the patient using the P_{RF} value and flip angle determined at step 202, i.e. in accordance with otherwise conventional techniques. If, however, the patient has an implant, then the Implant Mode is activated by the user and, at step 208, the MRI scaling controller inputs the R_{SAR} ratio (determined, e.g., at step 104 of FIG. 2) or other appropriate scaling factor derived from maxSARo and maxSARi (such as the “worst case” R_{SAR}_{_{—}}_{MAX}.) At step 210, the scaling controller scales the P_{RF} value based on R_{SAR}_{_{—}}_{MAX} to reduce P_{RF} or adjusts the flip angle to achieve corresponding or equivalent results. Then, at step 212, the MRI system images the patient using the reduced P_{RF} or adjusted flip angle.

### Device-Specific RF Power-Scaling Systems and Procedures

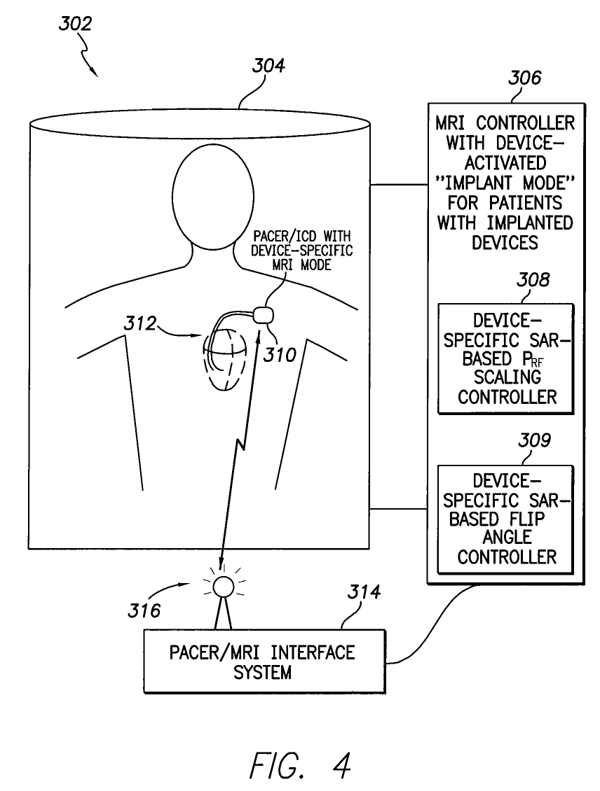

FIG. 4 illustrates an alternative MRI system 302 similar to the system of FIG. 1 but wherein the MRI system is equipped within an interface for receiving signals from the pacer/ICD implanted within the patient to be imaged. The signals are representative of device-specific data, which are then used by the MRI controller to determine an R_{SAR} value for use with the particular patient. The device-specific data can include, e.g., the make/model of the implanted device and the make/model of the lead system. Systems and techniques for transmitting data from a pacer/ICD to an MRI system are described in U.S. patent application Ser. No. 11/938,088, filed Nov. 9, 2007, entitled “Systems and Methods for Remote Monitoring of Signals Sensed by an Implantable Medical Device during an MRI.”

As with the system of FIG. 1, the MRI system of FIG. 4 includes an MRI machine 304 operating under the control of an MRI controller 306, which controls the strength and orientation of the fields generated during the MRI procedure. The patient has a pacer/ICD 310 implanted therein, along with leads 312. The Implant Mode of the MRI machine is equipped to store device-specific SAR data. The Implant Mode is activated by signals received from the implanted device through a pacer/MRI interface system 314. The interface system uses an antenna 316 to receive the data signals. Preferably, the pacer/ICD automatically switches into an internal MRI Mode upon entry into an MRI room and begins transmitting device-specific data. Automatic techniques for triggering transmission of data upon entry into an MRI room are also set forth in the above-cited patent application of Min et al. The interface then forwards the received data to the MRI controller. Alternatively, the personnel operating the MRI system can instead use an otherwise conventional external programmer device to retrieve data from the pacer/ICD of the patient for input into the MRI controller.

In any case, once the device-specific data is input into MRI controller 306, the data is used by a device-specific SAR-based P_{RF} scaling controller 308 to scale the RF power of the MRI for the patient based, at least in part, on the device-specific data transmitted by the pacer/ICD. That is, the scaling controller inputs the maxSARo and maxSARi values, which have been pre-determined using techniques described above, then scales the P_{RF} value for the patient based on maxSARo, maxSARi and the device-specific SAR data, before imaging the tissues of the patient at the reduced power level. In this manner, the scaling controller takes into account the device-specific data such as its make/model while determining the R_{SAR} value so as to produce an R_{SAR} value appropriate for the particular device. By generating a device-specific R_{SAR} value, P_{RF} typically need not be reduced as much as when using a “worst case” R_{SAR} values (i.e. R_{SAR}_{_{—}}_{MAX}.) Additionally or alternatively, the MRI controller includes a device-specific SAR-based flip angle controller 309, which calculates the device-specific R_{SAR} value then adjusts flip angles or flip angle sequences to achieve corresponding or equivalent results to that of RF power scaling.

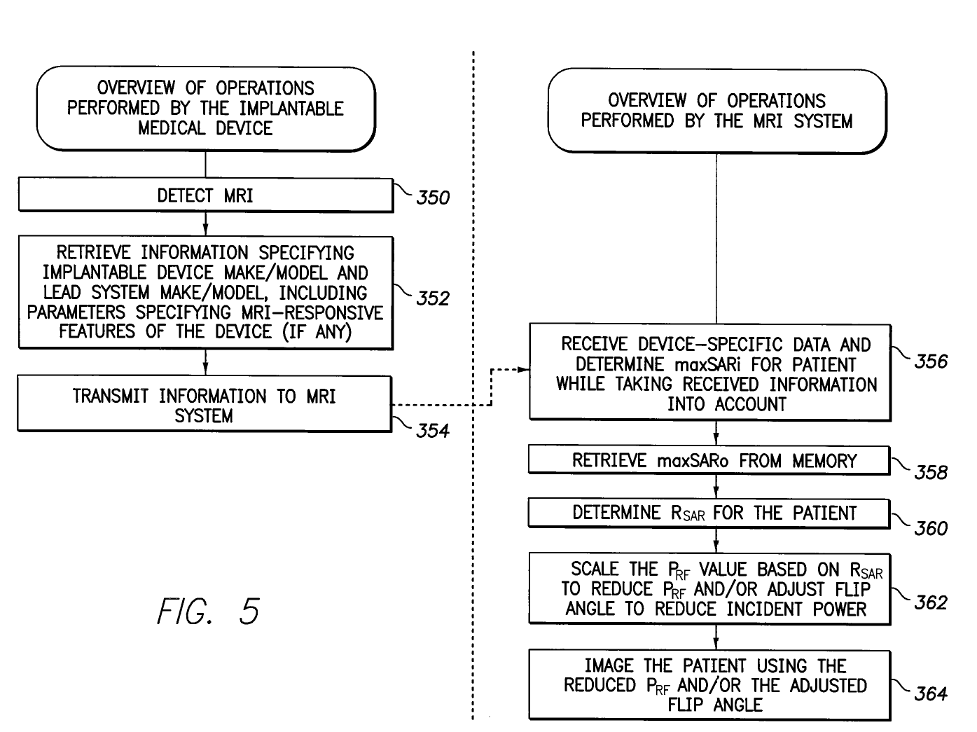

FIG. 5 summarizes a technique that may be performed using the system of FIG. 4. Operations performed by the pacer/ICD are shown on the left; whereas operations performed by the MRI system are shown on the right. At step 350, the pacer/ICD detects the MRI system (either automatically or via receipt of operator-initiated control signals received, for example, from an external programmer) and activates an MRI Mode where MRI-responsive techniques and features are activated. At step 352, the pacer/ICD retrieves any device-specific data stored within internal memory, such as, the make/model of the device and its leads. Other types of information that may be stored in device memory includes information pertaining to any passive or active MRI-responsive features of the implanted device and its leads. For example, if the leads are equipped with passive RF filters or active switches for reducing tip temperatures during MRIs, information identifying those features might be stored within device memory for subsequent use by the MRI system (or the personnel operating the MRI system) when determining the appropriate RF power to be used for the patient. In this regard, the maxSARi for a patient with an implanted device having MRI-responsive leads may be considerably higher than for a patient with an implanted device having conventional leads. Indeed, the more effective the MRI-responsive features of the implanted components, the more closely the maxSARi for the patient approaches maxSARo (i.e. the max local SAR for patients without implants).

MRI-responsive techniques and features are discussed in, e.g., the following patent applications: U.S. patent application Ser. No. 11/955,268, filed Dec. 12, 2007, of Min, entitled “Systems and Methods for Determining Inductance and Capacitance Values for use with LC Filters within Implantable Medical Device Leads to Reduce Lead Heating during an MRI”; U.S. patent application Ser. No. 11/963,243, filed Dec. 21, 2007, of Vase et al., entitled “MEMS-based RF Filtering Devices for Implantable Medical Device Leads to Reduce Lead Heating during MRI”; U.S. patent application Ser. No. 11/943,499, filed Nov. 20, 2007, of Zhao et al., entitled “RF Filter Packaging for Coaxial Implantable Medical Device Lead to Reduce Lead Heating during MRI” (Attorney Docket No. A07P1171); U.S. patent application Ser. No. 12/117,069, filed May 8, 2008, of Vase, entitled “Shaft-mounted RF Filtering Elements for Implantable Medical Device Lead to Reduce Lead Heating During MRI” (Attorney Docket No. A07e1005); U.S. patent application Ser. No. 11/860,342, filed Sep. 27, 2007, of Min et al., entitled “Systems and Methods for using Capacitive Elements to Reduce Heating within Implantable Medical Device Leads during an MRI”; U.S. patent application Ser. No. 12/042,605, filed Mar. 5, 2009, of Mouchawar et al., entitled “Systems and Methods for using Resistive Elements and Switching Systems to Reduce Heating within Implantable Medical Device Leads during an MRI” (Attorney Docket No. A08P1006); U.S. patent application Ser. No. 12/257,263, filed Oct. 23, 2008, of Min, entitled “Systems and Methods for Exploiting the Ring Conductor of a Coaxial Implantable Medical Device Lead to provide RF Shielding during an MRI to Reduce Lead Heating” (Attorney Docket No. A08P1048); U.S. patent application Ser. No. 12/257,245, filed Oct. 23, 2008, of Min, entitled “Systems and Methods for Disconnecting Electrodes of Leads of Implantable Medical Devices during an MRI to Reduce Lead Heating while also providing RF Shielding” (Attorney Docket No. A08P1049).

At step 354, the device-specific data is transmitted to the MRI system, either directly or via an external programmer or other intermediary device. In the example of FIG. 4, the interface system receives the data from the implanted device and forwards the data to the MRI controller to enable the device-activated Implant Mode. In other examples, the data is read from the implanted device using an external programmer then input to the MRI system, manually or automatically. In any case, at step 356, the MRI system receives the device-specific data and determines the maxSARi for the particular patient while taking the device-specific in to account. As noted, the patient-specific data can specify the model numbers of any implanted components. The MRI controller includes a database with lookup tables specifying predetermined maxSARi values to be used with patients with those particular components. In other cases, the lookup tables of the MRI controller include correction factors for adjusting a general maxSARi value (predetermined for an entire patient population) to yield an adjusted maxSARi or flip angle (appropriate to the particular patient.) In still other cases, lookup tables are not provided. Rather, the MRI controller is provided with conversion algorithms for converting an initial maxSARi (appropriate for a patient population) to an adjusted maxSARi (appropriate for the particular patient) based on the device-specific data. As noted, the more effective the MRI-responsive components of the implantable device, the more closely maxSARi for the patient approaches maxSARo. In any case, any data to be stored in lookup tables within the MRI controller (or any conversion algorithms to be used to convert an initial maxSARi to a device-specific maxSARi) is predetermined based on suitable experimentation and validation techniques. Typically, the data and/or conversion algorithms are validated in advance by the FDA or other appropriate government entity before employed for use with actual patients.

At step 358, the MRI controller then retrieves a previously determined maxSARo value from memory and, at step 360, determines the appropriate R_{SAR} value for the patient, using techniques already described. At step 362, the MRI controller then scales the P_{RF} value that would be used for the patient assuming no implants to a new P_{RF} value appropriate for the particular patient and/or adjusts flip angles or flip angle sequences to achieve corresponding or equivalent results. Since the maxSARi value determined at step 356 takes into account device-specific data, the scaled P_{RF} value likewise takes that information into account, thereby providing a scaled P_{RF} value that is more precisely optimized for the patient. At step 364, the MRI system then images the patient using the scaled (i.e. reduced) P_{RF} and/or the adjusted flip angles.

Hence, FIGS. 4-5 illustrate embodiments where device-specific information is stored within the pacer/ICD for use by the MRI controller to determine an appropriate P_{RF} value and/or flip angle for use in imaging the patient. In other examples, the device-specific data is not stored within the implanted device but is instead stored in a database that the MRI controller can access, such as a centralized database accessible via the Internet. As can be appreciated, a wide range of implementations are consistent with the general principles of the invention and all possible implementations are not described herein.

### Patient-Specific RF Power-Scaling Systems and Procedures

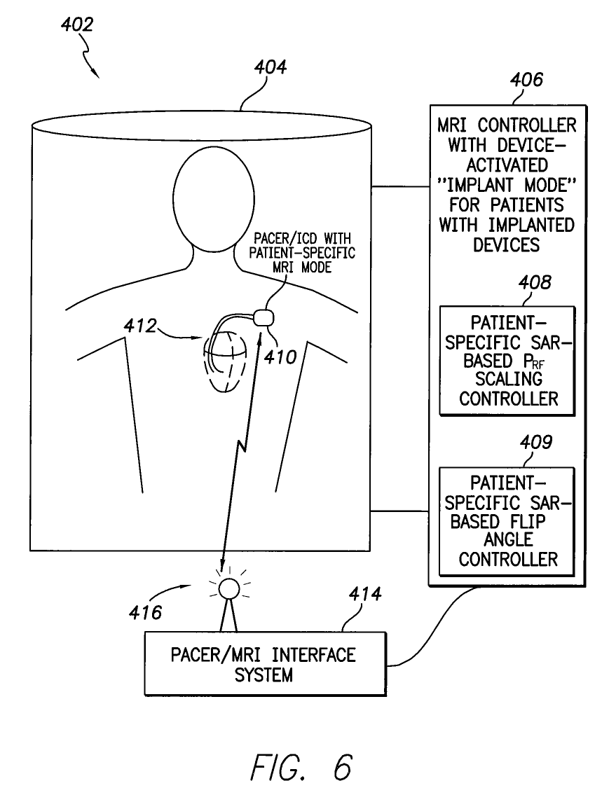

FIG. 6 illustrates an alternative MRI system 402 similar to the system of FIG. 4 but wherein the implantable device also transmits patient-specific data, which are then used by the MRI controller to determine a R_{SAR} value for use with the particular patient. The patient-specific data can include, e.g., a previously determined R_{SAR} value or preferred flip angle for the patient. As with the system of FIG. 4, the MRI system of FIG. 6 includes an MRI machine 404 operating under the control of an MRI controller 406, which controls the strength and orientation of the fields generated during the MRI procedure. The patient has a pacer/ICD 410 implanted therein, along with leads 412. Preferably, the pacer/ICD automatically switches into an MRI Mode upon entry into an MRI room and begins transmitting patient-specific data. The pacer/ICD transmits the patient-specific data to a pacer/MRI interface system 414 having an antenna 416 for input to MRI controller 406.

Once the patient-specific data is input into MRI controller 406, the data is used by a device-specific SAR-based P_{RF} scaling controller 408 to scale the RF power of the MRI for the patient based, at least in part, on the patient-specific data transmitted by the pacer/ICD. That is, the scaling controller inputs maxSARo and maxSARi values, which have been pre-determined using techniques described above, then scales the P_{RF} value for the patient based on maxSARo, maxSARi and the patient-specific SAR data before imaging the tissues of the patient at the reduced power level. That is, the controller takes into account the patient-specific data while determined the R_{SAR} value so as to produce an R_{SAR} value appropriate for the particular patient. By generating a patient-specific R_{SAR} value, P_{RF} typically need not be reduced as much as when using a “worst case” R_{SAR} values (i.e. R_{SAR}_{_{—}}_{MAX}.) Additionally or alternatively, the MRI controller includes a device-specific SAR-based flip angle controller 409, which calculates the device-specific R_{SAR} value then adjusts flip angles or flip angle sequences to achieve corresponding or equivalent results to that of RF power scaling.

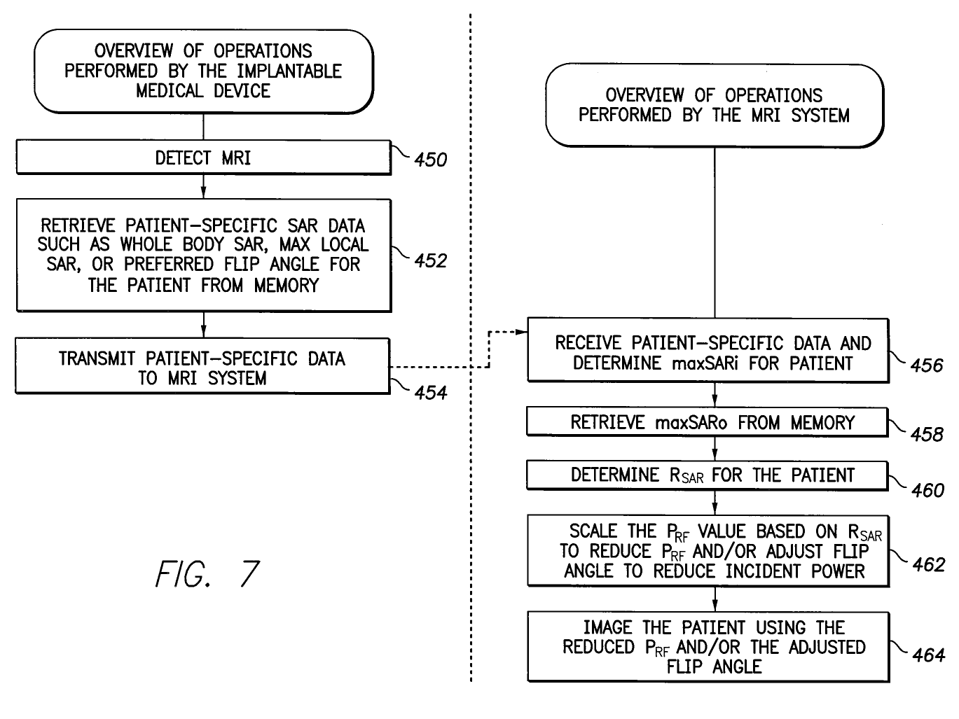

FIG. 7 summarizes a technique that may be performed using the system of FIG. 6. Operations performed by the pacer/ICD are again shown on the left; whereas operations performed by the MRI system are shown on the right. At step 450, the pacer/ICD detects the MRI system and, at step 452, retrieves any patient-specific SAR data stored within internal memory, such as, the maximum local SAR for the patient (i.e. the maxSARi for the particular patient), and/or the preferred flip angle to be used for the patient. Note that the maximum local SAR for the particular patient (i.e. maxSARi for the patient) may be determined in advance using otherwise conventional techniques, then programmed into device memory. In some implementations, if the patient has been subject to a previous MRI, any patient-specific data obtained at that time is stored within the memory of the pacer/ICD such that, if another MRI procedure is required, that information can be readily retrieved from the device and need not be re-determined.

As noted, one particular value that may be stored by the device and then exploited by the MRI system is the preferred flip angle for use with the patient and any particular selected MRI sequences. The flip angle α relates to the angle of excitation for a field echo pulse sequence of an MRI. That is, α is the angle through which the net magnetization is rotated or tipped relative to the main magnetic field direction via the application of a RF excitation pulse at the Larmor frequency. As such, the RF power of the pulse is proportional to the particular flip angle through which the spins are tilted under the influence of the magnetic fields. Flip angle is sometimes also referred to as the tip angle, nutation angle or angle of nutation. Flip angles between 0° and 90° are typically used in gradient echo sequences. A series of 180° pulses are typically used in spin echo sequences. An initial 180° pulse followed by a 90° pulse and a 180° pulse are typically used in inversion recovery sequences. However, in some cases, a particular flip angle adjustment might be preferred for achieving the same scaling effect of P_{RF} for a particular patient/MRI imaging sequence.

At step 454, the patient-specific data is transmitted to the MRI system. In the example of FIG. 6, the interface system receives the data from the implanted device and forwards the data to the MRI controller to enable the Implant Mode. At step 456, the MRI system receives the patient-specific data and determines the maxSARi for the particular patient. If the patient-specific data already specifies the maxSARi for the patient then, of course, the MRI controller merely reads out that data. Otherwise, the MRI controller processes the data received to determine the appropriate maxSARi for the patient. In some examples, lookup tables of the MRI controller include correction factors for adjusting a general maxSARi value (predetermined for an entire patient population) to yield an adjusted maxSARi or flip angle (appropriate to the particular patient.) In still other cases, lookup tables are not provided. Rather, the MRI controller is provided with conversion algorithms for converting an initial maxSARi (appropriate for a patient population) to an adjusted maxSARi (appropriate for the particular patient) based on the patient-specific data. In any case, any data to be stored in lookup tables within the MRI controller (or any conversion algorithms to be used to convert an initial maxSARi to a device-specific maxSARi) is predetermined based on suitable experimentation and validation techniques. Typically, the data and/or conversion algorithms are validated in advance by the FDA or other appropriate government entity before employed for use with actual patients.

At step 458, the MRI controller then retrieves a previously determined maxSARo value from memory and, at step 460, determines the appropriate R_{SAR} value for the patient, using techniques already described. At step 462, the MRI controller then scales the P_{RF} value that would be used for the patient assuming no implants to a new P_{RF} value appropriate for the particular patient. Since the maxSARi value determined at step 456 takes into account patient-specific data, the scaled P_{RF} value likewise takes that information into account, thereby providing a scaled P_{RF} value that is more precisely optimized for the particular patient. Additionally or alternatively, flip angles or flip angle sequences are adjusted to achieve corresponding or equivalent results to that of RF power scaling. At step 464, the MRI system then images the patient.

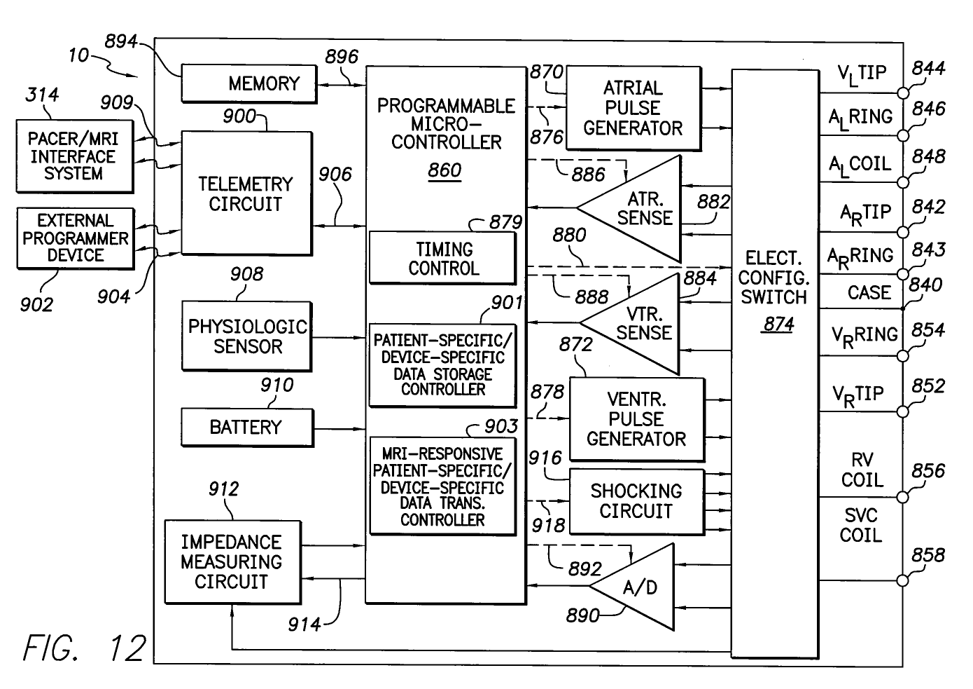

Hence, FIGS. 6-7 illustrate embodiments where patient-specific information is stored within the pacer/ICD for use by the MRI controller to determine an appropriate P_{RF} value for the patient. As with device-specific data, patient-specific data can instead be stored in a database that the MRI controller accesses, such as a centralized database accessible via the Internet. Also, although an example has been described where the controller uses the patient-specific data to determine maxSARi and then R_{SAR}, other procedures may instead be used. In some cases, for example, the patient-specific data specifies the preferred R_{SAR} value to be used (or specifies data from which R_{SAR} can be determined without first determining maxSARi value for the patient). In other cases, for example, the patient-specific data specifies the preferred P_{RF} value to be used (or specifies data from which P_{RF} can be determined without first determining either maxSARi or R_{SAR} for the patient). In particular, if the patient has been subject to a prior MRI using a similar machine, any data pertinent to that MRI session can be stored within the pacer/ICD (or elsewhere) to facilitate a subsequent MRI procedure. Implementations exploiting both patient-specific and device-specific data can be employed (as shown in FIG. 12, discussed below.) As can be appreciated, a wide range of implementations are consistent with the general principles of the invention and all possible implementations are not described herein.

### Materials-Based RF Power Reduction Systems and Procedures

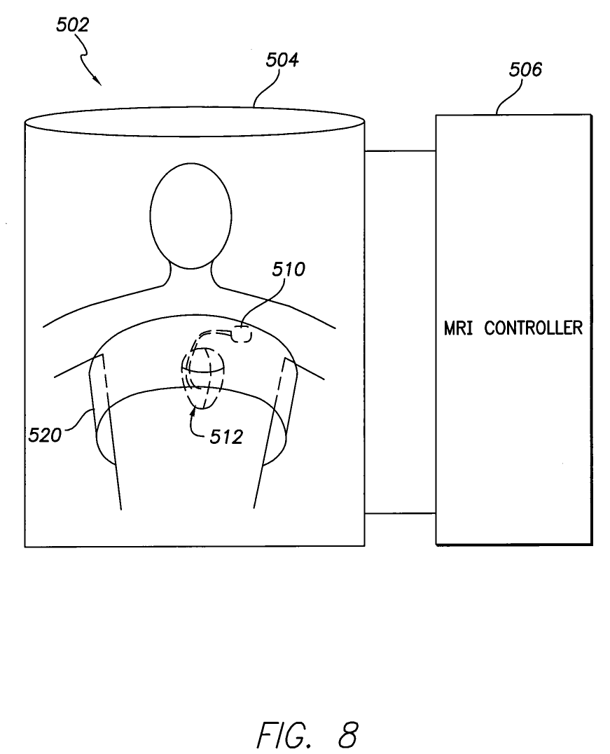

FIG. 8 illustrates an otherwise conventional MRI system 402 wherein the MRI system is not necessarily equipped to automatically scale RF power using the R_{SAR}-based techniques described above. Rather, an article 520, such as a blanket, jacket, pad or the like, is placed around the patient. The article 520 is formed of dielectric or conductive/resistive materials capable of attenuating RF signals so as to reduce the strength of the signals radiating the tissues of the patient. As with the previously described MRI systems, the system of FIG. 8, includes an MRI machine 504 operating under the control of an MRI controller 506, which controls the strength and orientation of the fields generated by the MRI. The patient has a pacer/ICD 510 implanted therein, along with leads 512. However, the MRI controller does not include a SAR-based power-scaling controller or a SAR-based flip angle controller as in the previously described embodiments. Rather, the MRI controller determines and uses P_{RF} values and flip angles for the patient as if no implant were present, i.e. based on maxSARo. Operators of the MRI system select one or more RF power-attenuating articles for placement on, under or around the patient. The particular materials to be use may be selected, at least in part, based on R_{SAR} (either derived for an entire patient population as in FIGS. 1-3 or derived for the particular patient as in FIGS. 6-7) so as to reduce the RF fields within the patient in the vicinity of the pacer/ICD to be less than maxSARi.

In the illustration of FIG. 8, the RF power-attenuating article 514 is generally cylindrical and is wrapped or positioned around the torso of the patient so as to attenuate RF power in the vicinity of the pacer/ICD. This is merely a stylized illustration. In practice, the article may need to be fitted more closely around the torso of the patient and, depending upon the needs of the patient, may need to cover more or less surface area. Otherwise routine experimentation may be performed to determine optimum sizes, arrangements and positions for such articles so as to achieve a desired amount of RF power attenuation. In one example, the articles are formed of such materials as saline or gel filled jacket with adjustable dielectric constant and conductivities, which are known to attenuate RF fields. The thickness of the particular article to be used on a given patient depends on the material used to form the article, as well as on the amount of RF power attenuation required for that patient.

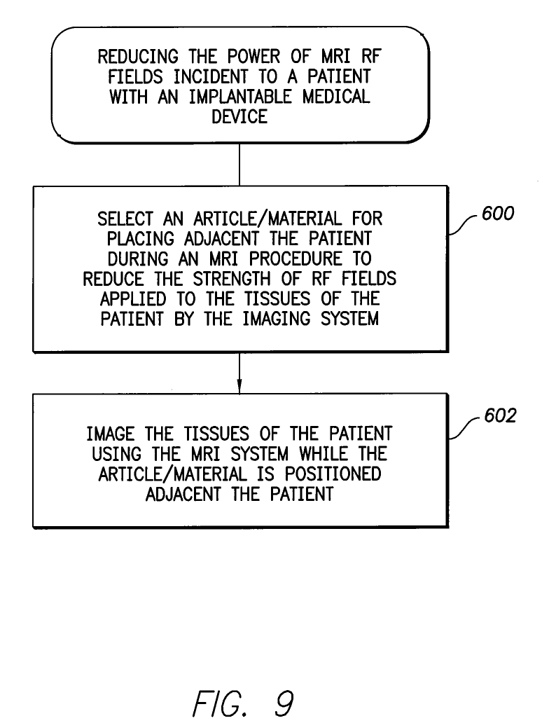

FIG. 9 broadly summarizes the RF attenuation technique performed while using the system of FIG. 8. At step 600, an article/material is selected for placing adjacent the patient during an MRI procedure to reduce the strength of RF fields applied to the tissues of the patient by the imaging system. At step 602, the tissues of the patient are imaged using the MRI system while the article/material is positioned adjacent the patient.

In a typical implementation, a single article is provided for use with MRI systems that has sufficient RF power attenuation capability to be safely used with any patient with implants. That is, the article is designed to be safe and effective for patients with implants even in the “worst case” scenario, either for all MRI machines or for classes or MRI machines. The optimal thickness and shape of the article is ascertained in advance using test phantoms while taking into account maxSARi and maxSARo, then validated with the FDA or other appropriate government entity for use with actual patients. As such, the operators of a given MRI system need not select among different articles of differing thickness. Rather, the operators need only determine whether a patient to be scanned has an implant and, if so, the article is placed around the patient in the vicinity of the implant to attenuate RF power. By providing a single article validated for the “worst case” scenario, no special expertise is required to accommodate patients with implants.

In other implementations, however, a selection of articles of differing materials, shapes or thicknesses may be supplied. The operators of the MRI system select the particular article or articles to be used with a given patient based on patient-specific data such as the maxSARi for the patient, whether the implanted device of the patient has MRI-responsive features. As noted, such data may be stored within the device itself (and accessed via an external programmer) or may be available via a centralized database. In any case, the operators of the MRI system then select the appropriate articles to be used for each individual patient, so as to achieve the least amount of RF attenuation (so as to achieve the best MRI images) while still ensuring the safety of the patient.

Insofar as jackets are concerned, the information needed by the operators to choose the correct jacket is preferably imprinted on the jacket. In another example, the operators are provided with lookup tables that specify particular models of implantable devices and leads, along with the appropriate thickness of RF attenuation materials to be used for patients with those devices/leads. The operator then merely looks up the appropriate thickness to be used based on the particular components implanted within the patient and selects the RF attenuation materials accordingly. In any case, any data to be stored in lookup tables provided to the operators is predetermined based on suitable experimentation and validation techniques. Typically, the lookup table data and the articles/materials to be used are validated in advance by the FDA or other appropriate government entity before employed for use with actual patients with implants.

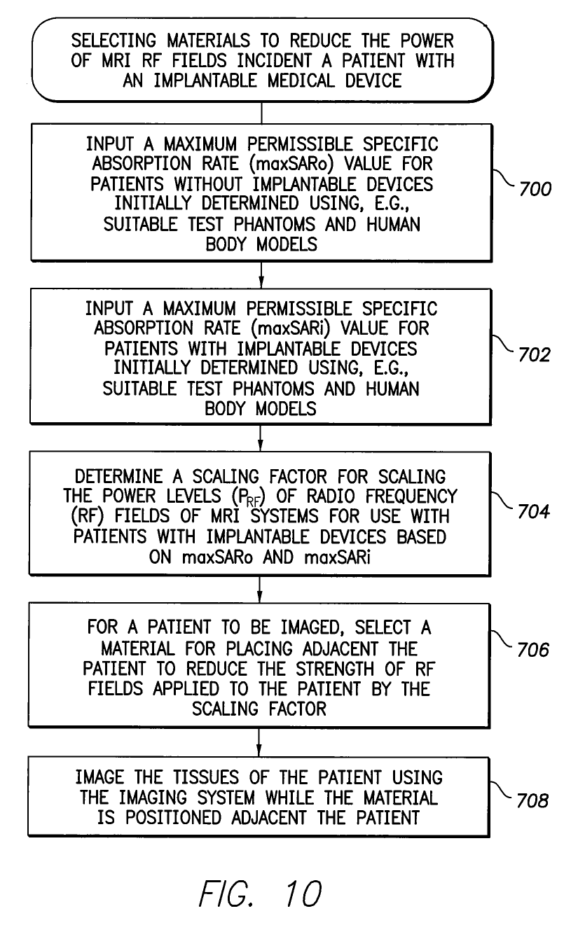

FIG. 10 provides a more detailed example of the general technique of FIG. 9, wherein the articles/materials are selected based on the aforementioned R_{SAR} scaling factor. Briefly, beginning at step 700, maxSARo is determined for patients without implantable devices, such as with reference to FDA or other appropriate government agency guidelines. At step 702, maxSARi is determined for patients with implantable devices, as discussed above. At step 704, the R_{SAR} scaling factor is determined for scaling the power levels of RF fields of the MRI system for use with patients with implantable devices based on maxSARo and maxSARi. As above, R_{SAR} is preferably calculated as the ratio of maxSARo/maxSARi. If different maxSARi and maxSARo values are specified for different populations of patients, then R_{SAR} is calculated based on the particular maxSARi and maxSARo values appropriate for the particular patient. At step, 706, for the patient to be imaged, a material is selected for placing adjacent the patient to reduce the strength of RF fields applied to the patient by the scaling factor. That is, one or more articles/materials are selected that will achieve the requisite amount of power reduction specified by the scaling factor. At step 708, the MRI system then images the tissues of the patient using the MRI at its normal power level, but with the articles/materials positioned around the portions of the patient containing implanted devices so as to attenuate the RF fields to reduce the local SAR within tissues of the patient near the implanted deice to below maxSARi.

In this manner, as with the preceding implementations, the MRI system can use procedures previously validated by the FDA (or other appropriate government entity) to initially determine the appropriate P_{RF} value for the patient, without regard to the presence of the implanted device. Then, the RF fields are attenuated by the effect of the articles/materials, yielding reduced RF fields within the tissues of the patient near the implanted device, so as to be safe for the patient, despite the presence of the device. Although FIG. 10 illustrates an example wherein R_{SAR} is explicitly calculated, other procedures may be performed that do not require explicit calculation of R_{SAR} prior to selection of articles/materials for reducing RF power within the patient. As can be appreciated, a wide range of implementations are consistent with the general principles of the invention and all possible implementations are not described herein.

The techniques discussed above can be implemented in connection with a wide variety of implantable medical devices for use with a wide variety of MRI systems. For the sake of completeness, a detailed description of an exemplary pacer/ICD will now be provided.

### Exemplary Pacer/ICD

With reference to FIGS. 11 and 12, a description of the pacer/ICD of FIG. 6 will now be provided. FIG. 11 provides a simplified diagram of the pacer/ICD, which is a dual-chamber stimulation device capable of treating both fast and slow arrhythmias with stimulation therapy, including cardioversion, defibrillation, and pacing stimulation, as well as capable of storing patient-specific MRI data.

To provide atrial chamber pacing stimulation and sensing, pacer/ICD 310 is shown in electrical communication with a heart 812 by way of a left atrial lead 820 having an atrial tip electrode 822 and an atrial ring electrode 823 implanted in the atrial appendage. Pacer/ICD 310 is also in electrical communication with the heart by way of a right ventricular lead 830 having, in this embodiment, a ventricular tip electrode 832, a right ventricular ring electrode 834, a right ventricular (RV) coil electrode 836, and a superior vena cava (SVC) coil electrode 838. Typically, the right ventricular lead 830 is transvenously inserted into the heart so as to place the RV coil electrode 836 in the right ventricular apex, and the SVC coil electrode 838 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 310 is coupled to a “coronary sinus” lead 824 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 824 is designed to receive atrial and ventricular cardiac signals and to deliver left ventricular pacing therapy using at least a left ventricular tip electrode 826, left atrial pacing therapy using at least a left atrial ring electrode 827, and shocking therapy using at least a left atrial coil electrode 828. With this configuration, biventricular pacing can be performed. Although only three leads are shown in FIG. 11, it should also be understood that additional stimulation leads (with one or more pacing, sensing and/or shocking electrodes) may be used in order to efficiently and effectively provide pacing stimulation to the left side of the heart or atrial cardioversion and/or defibrillation.

A simplified block diagram of internal components of pacer/ICD 310 is shown in FIG. 12. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation as well as providing for the aforementioned apnea detection and therapy.

The housing 840 for pacer/ICD 310, shown schematically in FIG. 12, is often referred to as the “can”, “case” or “case electrode” and may be programmably selected to act as the return electrode for all “unipolar” modes. The housing 840 may further be used as a return electrode alone or in combination with one or more of the coil electrodes, 828, 836 and 838, for shocking purposes. The housing 840 further includes a connector (not shown) having a plurality of terminals, 842, 843, 844, 846, 848, 852, 854, 856 and 858 (shown schematically and, for convenience, the names of the electrodes to which they are connected are shown next to the terminals). As such, to achieve right atrial sensing and pacing, the connector includes at least a right atrial tip terminal (A_{R} TIP) 842 adapted for connection to the atrial tip electrode 822 and a right atrial ring (A_{R} RING) electrode 843 adapted for connection to right atrial ring electrode 823. To achieve left chamber sensing, pacing and shocking, the connector includes at least a left ventricular tip terminal (V_{L} TIP) 844, a left atrial ring terminal (A_{L} RING) 846, and a left atrial shocking terminal (A_{L} COIL) 848, which are adapted for connection to the left ventricular ring electrode 826, the left atrial tip electrode 827, and the left atrial coil electrode 828, respectively. To support right chamber sensing, pacing and shocking, the connector further includes a right ventricular tip terminal (V_{R} TIP) 852, a right ventricular ring terminal (V_{R} RING) 854, a right ventricular shocking terminal (R_{V} COIL) 856, and an SVC shocking terminal (SVC COIL) 858, which are adapted for connection to the right ventricular tip electrode 832, right ventricular ring electrode 834, the RV coil electrode 836, and the SVC coil electrode 838, respectively.

At the core of pacer/ICD 310 is a programmable microcontroller 860, which controls the various modes of stimulation therapy. As is well known in the art, the microcontroller 860 (also referred to herein as a control unit) typically includes a microprocessor, or equivalent control circuitry, designed specifically for controlling the delivery of stimulation therapy and may further include RAM or ROM memory, logic and timing circuitry, state system circuitry, and I/O circuitry. Typically, the microcontroller 860 includes the ability to process or monitor input signals (data) as controlled by a program code stored in a designated block of memory. The details of the design and operation of the microcontroller 860 are not critical to the invention. Rather, any suitable microcontroller 860 may be used that carries out the functions described herein. The use of microprocessor-based control circuits for performing timing and data analysis functions are well known in the art.

As shown in FIG. 12, an atrial pulse generator 870 and a ventricular pulse generator 872 generate pacing stimulation pulses for delivery by the right atrial lead 820, the right ventricular lead 830, and/or the coronary sinus lead 824 via an electrode configuration switch 874. It is understood that in order to provide stimulation therapy in each of the four chambers of the heart, the atrial and ventricular pulse generators, 870 and 872, may include dedicated, independent pulse generators, multiplexed pulse generators or shared pulse generators. The pulse generators, 870 and 872, are controlled by the microcontroller 860 via appropriate control signals, 876 and 878, respectively, to trigger or inhibit the stimulation pulses.

The microcontroller 860 further includes timing control circuitry (not separately shown) used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 874 includes a plurality of switches for connecting the desired electrodes to the appropriate I/O circuits, thereby providing complete electrode programmability. Accordingly, the switch 874, in response to a control signal 880 from the microcontroller 860, determines the polarity of the stimulation pulses (e.g., unipolar, bipolar, combipolar, etc.) by selectively closing the appropriate combination of switches (not shown) as is known in the art.

Atrial sensing circuits 882 and ventricular sensing circuits 884 may also be selectively coupled to the right atrial lead 820, coronary sinus lead 824, and the right ventricular lead 830, through the switch 874 for detecting the presence of cardiac activity in each of the four chambers of the heart. Accordingly, the atrial (ATR. SENSE) and ventricular (VTR. SENSE) sensing circuits, 882 and 884, may include dedicated sense amplifiers, multiplexed amplifiers or shared amplifiers. The switch 874 determines the “sensing polarity” of the cardiac signal by selectively closing the appropriate switches, as is also known in the art. In this way, the clinician may program the sensing polarity independent of the stimulation polarity. Each sensing circuit, 882 and 884, preferably employs one or more low power, precision amplifiers with programmable gain and/or automatic gain control, bandpass filtering, and a threshold detection circuit, as known in the art, to selectively sense the cardiac signal of interest. The automatic gain control enables pacer/ICD 310 to deal effectively with the difficult problem of sensing the low amplitude signal characteristics of atrial or ventricular fibrillation. The outputs of the atrial and ventricular sensing circuits, 882 and 884, are connected to the microcontroller 860 which, in turn, are able to trigger or inhibit the atrial and ventricular pulse generators, 870 and 872, respectively, in a demand fashion in response to the absence or presence of cardiac activity in the appropriate chambers of the heart.

For arrhythmia detection, pacer/ICD 310 utilizes the atrial and ventricular sensing circuits, 882 and 884, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 860 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 890. The data acquisition system 890 is configured to acquire intracardiac electrogram signals, convert the raw analog data into a digital signal, and store the digital signals for later processing and/or telemetric transmission to an external device 902. The data acquisition system 890 is coupled to the right atrial lead 820, the coronary sinus lead 824, and the right ventricular lead 830 through the switch 874 to sample cardiac signals across any pair of desired electrodes. The microcontroller 860 is further coupled to a memory 894 by a suitable data/address bus 896, wherein the programmable operating parameters used by the microcontroller 860 are stored and modified, as required, in order to customize the operation of pacer/CD 310 to suit the needs of a particular patient. Such operating parameters define, for example, pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 310 may be non-invasively programmed into the memory 894 through a telemetry circuit 900 in telemetric communication with an external device 902, such as a programmer, transtelephonic transceiver or a diagnostic system analyzer, or the pacer/MRI interface system 314 (FIG. 6). The telemetry circuit 900 is activated by the microcontroller by a control signal 906. The telemetry circuit 900 advantageously allows IEGMs and other electrophysiological signals and/or hemodynamic signals, and status information relating to the operation of pacer/ICD 310 (as stored in the microcontroller 860 or memory 894) to be sent to the external programmer device 902 through an established communication link 904 or to a separate interface system via link 909.

Pacer/ICD 310 further includes an accelerometer or other physiologic sensor 908, commonly referred to as a “rate-responsive” sensor because it is typically used to adjust pacing stimulation rate according to the exercise state of the patient. However, the physiological sensor 908 may further be used to detect changes in cardiac output, changes in the physiological condition of the heart, or diurnal changes in activity (e.g., detecting sleep and wake states) and to detect arousal from sleep. Accordingly, the microcontroller 860 responds by adjusting the various pacing parameters (such as rate, AV Delay, V-V Delay, etc.) at which the atrial and ventricular pulse generators, 870 and 872, generate stimulation pulses. While shown as being included within pacer/ICD 310, it is to be understood that the physiologic sensor 908 may also be external to pacer/ICD 310, yet still be implanted within or carried by the patient, such as sensor 837 of FIG. 12. A common type of rate responsive sensor is an activity sensor incorporating an accelerometer or a piezoelectric crystal, which is mounted within the housing 840 of pacer/ICD 310. Other types of physiologic sensors are also known, for example, sensors that sense the oxygen content of blood, respiration rate and/or minute ventilation, pH of blood, ventricular gradient, etc.

The pacer/ICD additionally includes a battery 910, which provides operating power to all of the circuits shown in FIG. 12. The battery 910 may vary depending on the capabilities of pacer/ICD 310. If the system only provides low voltage therapy, a lithium iodine or lithium copper fluoride cell may be utilized. For pacer/ICD 310, which employs shocking therapy, the battery 910 must be capable of operating at low current drains for long periods, and then be capable of providing high-current pulses (for capacitor charging) when the patient requires a shock pulse. The battery 910 must also have a predictable discharge characteristic so that elective replacement time can be detected. Accordingly, pacer/ICD 310 is preferably capable of high voltage therapy and appropriate batteries.

As further shown in FIG. 12, pacer/ICD 310 is shown as having an impedance measuring circuit 912 which is enabled by the microcontroller 860 via a control signal 914. Herein, thoracic impedance is primarily detected for use in tracking thoracic respiratory oscillations. Other uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, etc. The impedance measuring circuit 120 is advantageously coupled to the switch 74 so that any desired electrode may be used.

In the case where pacer/ICD 310 is intended to operate as an ICD, it detects the occurrence of an arrhythmia, and automatically applies an appropriate electrical shock therapy to the heart aimed at terminating the detected arrhythmia. To this end, the microcontroller 860 further controls a shocking circuit 916 by way of a control signal 918. The shocking circuit 916 generates shocking pulses of low (up to 0.5 joules), moderate (0.5-10 joules) or high energy (11 to 40 joules), as controlled by the microcontroller 860. Such shocking pulses are applied to the heart of the patient through at least two shocking electrodes, and as shown in this embodiment, selected from the left atrial coil electrode 828, the RV coil electrode 836, and/or the SVC coil electrode 838. The housing 840 may act as an active electrode in combination with the RV electrode 836, or as part of a split electrical vector using the SVC coil electrode 838 or the left atrial coil electrode 828 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 11-40 joules), delivered asynchronously (since R-waves may be too disorganized), and pertaining exclusively to the treatment of fibrillation. Accordingly, the microcontroller 860 is capable of controlling the synchronous or asynchronous delivery of the shocking pulses.

Insofar as MRI-mode operations are concerned, the microcontroller includes an patient-specific/device-specific data storage controller 901, which is operative to control the storage and retrieval of patient-specific and/or device-specific data relevant to the determination of the appropriate RF power to be used during an MRI procedure, such as whole body SAR, max local SAR, preferred flip angle, etc., and/or information specifying any MRI-responsive features of the pacer/ICD and its leads, such as RF filters, switches, etc., generally in accordance with the techniques described above in connection with FIGS. 4-7. The microcontroller also includes an MRI-responsive patient-specific/device-specific data transmission controller 903, which is operative to control transmission of the patient-specific data and/or device-specific data to the pacer/MRI interface system (e.g. device 314 of FIG. 4) prior to a MRI procedure, generally in accordance with the techniques described above in connection with FIG. 5.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

What have been described are various systems and methods for reducing RF power within a patient during an MRI, particularly for use when imaging patients with pacer/ICDs or other implantable cardiac rhythm management devices. Principles of the invention may be exploiting using other implantable systems such as neural stimulators, other imaging systems generating strong RF fields, or in accordance with other techniques. Thus, while the invention has been described with reference to particular exemplary embodiments, modifications can be made thereto without departing from the scope of the invention.

What is claimed is:

1. A method for use in controlling magnetic resonance imaging (MRI) systems for imaging the tissues of patients with implantable medical devices, the method comprising:
determining a scaling factor for scaling the power levels (P_{RF}) of radio-frequency (RF) fields of MRI systems for use with patients with implantable devices, wherein the scaling factor is based on specific absorption rate (SAR) values for patients with implantable devices and SAR values for patients without implantable devices;
for patients with implantable devices, reducing the power level (P_{RF}) of the RF fields to be used based on the scaling factor; and
imaging the tissues of the patient using the MRI system at the reduced power level (P_{RF}).

2. The method of claim 1 wherein determining the scaling factor includes:
inputting a maximum SAR value for patients without implantable devices (maxSARo);
inputting a maximum SAR value for patients with implantable devices (maxSARi); and
determining the scaling factor based on a ratio (R_{SAR}) of the maximum SAR value for patients without implantable devices (maxSARo) to the maximum SAR value for patients with implantable devices (maxSARi).

3. The method of claim 2 wherein the maximum SAR values are local SAR values.

4. The method of claim 2 wherein the maxSARi value is a worst-case value (maxSARi_max) representative of the clinically relevant worst case and wherein the ratio value (R_{SAR}) is likewise representative of the clinically relevant worst case (R_{SAR}_{_{—}}_{MAX}).

5. A method for use in controlling magnetic resonance imaging (MRI) systems for imaging the tissues of patients with implantable medical devices, the method comprising:
determining a scaling factor for adjusting flip angles of magnetic fields of MRI systems for use with patients with implantable devices, wherein the scaling factor is based on specific absorption rate (SAR) values for patients with implantable devices and SAR values for patients without implantable devices;
for patients with an implantable device, adjusting flip angles to be used based on the scaling factor; and
imaging the tissues of the patient using the MRI system at the adjusted flip angles.

6. A method for use by a magnetic resonance imaging (MRI) system for imaging the tissues of a patient with an implantable medical device, the method comprising:
determining a power level for the radio-frequency (RF) fields of the MRI system for use in imaging the patient, without regard to the presence of the implantable medical device within the patient;
inputting a value representative of a ratio of a local specific absorption rate (SAR) value for patients without implantable devices to a local SAR value for patients with implantable devices;
scaling the power level of the RF fields of the MRI system based on the ratio; and
imaging the tissues of the patient using the MRI system at the scaled power level.

7. The method of claim 6 wherein scaling the power level (P_{RF}) of the RF fields of the MRI based on R_{SAR} includes reducing P_{RF} by the R_{SAR} ratio so that the patient receives an amount of power consistent with maxSARi.

8. The method of claim 7 wherein the maxSARi value is a worst-case value (maxSARi_max) representative of the clinically relevant worst case and wherein the ratio value (R_{SAR}_{_{—}}_{MAX}) is likewise representative of the clinically relevant worst case.

9. A method for use by a magnetic resonance imaging (MRI) system for imaging the tissues of a patient with an implantable medical device, the method comprising:
determining a flip angle for the magnetic fields of the MRI system for use in imaging the patient, without regard to the presence of the implantable medical device within the patient;
inputting a value representative of a ratio of a local specific absorption rate (SAR) value for patients without implantable devices to a local SAR value for patients with implantable devices;
adjusting the flip angle of the magnetic fields of the MRI system based on the ratio; and
imaging the tissues of the patient using the MRI system using the adjusted flip angle.

10. A method for use by an implantable medical device implanted within a patient, the method comprising:
storing values in the device relevant to the maximum specific absorption rate (SAR) of the patient; and
transmitting the values to an external system prior to a magnetic resonance imaging (MRI) procedure such that an MRI system performing the procedure can adjust its operation based, at least in part, on the transmitted values.

11. The method of claim 10 wherein the values relevant to the maximum SAR of the patients include one or more of: a predetermined maximum local SAR value for the patient, a predetermined scaling factor (R_{SAR}) for use with the patient, a predetermined flip angle for use with the patient, information pertaining to the implantable device, and information pertaining to any leads of the implantable device.

12. The method of claim 11 wherein the information pertaining to the implantable device includes information representative of any MRI-responsive functionality of the implantable system.

13. The method of claim 10 further including receiving the transmitted values using the external system and displaying the transmitted values using a display system of the external system.

14. A method for use with a magnetic resonance imaging (MRI) system for imaging the tissues of a patient having an implantable medical device, the method comprising:
selecting an radio-frequency (RF) power attenuating material for placing adjacent the patient during an MRI procedure to reduce the strength of RF fields generated within tissues of the patient by the MRI system; and
imaging the tissues of the patient using the MRI system while the material is positioned adjacent the patient.

15. The method of claim 14 wherein the materials are placed external to the patient at a location adjacent the implanted device within the patient.

16. The method of claim 15 wherein the medical device is implanted within the chest of the patient and wherein the materials are placed around the chest of the patient.

17. The method of claim 14 wherein selecting a material for placing adjacent the patient during an MRI procedure includes:
determining a local specific absorption rate (SAR) value for patients without implantable devices;
determining a local SAR value for patients with implantable devices;
determining a scaling factor for scaling the power levels (P_{RF}) of RF fields the MRI based on the maximum local SAR value for patients without implantable device and the maximum local SAR value for patients without implantable devices;
selecting the material for placing adjacent the patient during an MRI procedure based on the scaling factor.

18. An article for use with a magnetic resonance imaging (MRI) system for imaging the tissues of a patient with an implantable medical device, the article comprising:
a radio-frequency (RF) power attenuation material fitted to be placed adjacent the patient during an MRI procedure to reduce the strength of fields applied to the tissues of the patient by the MRI system;
wherein the material is formed from one or more of a conductive material, a resistive material and a dielectric material.

19. The article of claim 18 wherein the RF power attenuation material is configured as one or more of a jacket, a blanket, a pad or a wrap.

20. The article of claim 18 wherein a plurality of said materials are provided.
